(19) United States
(12) Patent Application Publication
(10) Pub. No.: US 2004/0254548 A1
(43) Pub. Date: Dec. 16, 2004

(54) Device for resorption of urine for male incontinence

(76) Inventor: Hans E. Sachse, Nuernberg (DE)

Correspondence Address: Herbert B. Keil, KEIL & WEINKAUF, 1350 Connecticut Ave., N.W., Washington, DC, 20036, US

(21) Appl. No.: 10/341,474

(22) Filed: Jan. 14, 2003

(30) Foreign Application Priority Data
Jan. 17, 2002 (DE) 102 01 524.4

(51) Int. Cl. A61F 5/44

(52) U.S. Cl. 604/349

(57) ABSTRACT

The invention relates to an incontinence insert for absorbing urine in male incontinence. This incontinence insert is placed in the genital region, between the underpants and the abdominal wall, and detachably joined to the underpants. Its shaping prevents the penis from coming to rest beside the incontinence insert. For this purpose the incontinence insert has two lateral peripheral beads, which fix the penis and thus the urethral orifice in the middle position. A further variant comprises the provision that the incontinence insert together with the underpants forms a functional unit, which has a slit in its middle region.

With increasing life expectancy, the number of incontinent persons is rising steadily from year to year; in men, it is also increasing after prostate operations.—The condition of urinary incontinence means skin irritation, soiled underwear, and odors: a major burden for the sufferer himself, but also for those around him.—To keep the area around the urethral orifice and the underwear near it dry, the only options are to insert a indwelling catheter, with the risk of an often life-threatening infection, to use a urine-absorbing pad known as an incontinence insert, or to use especially manufactured and very expensive urine-absorbing underwear.—The urine-absorbing pads—also called incontinence inserts—now on the market basically comprise two components: a urine-absorbing component facing the body, and a component impermeable to liquid facing the underwear or underpants. Normally the two components are joined solidly together to make a unit, and the part that is impermeable to liquid can be affixed detachably to the underwear by means of adhesive faces.—For wearing comfort and effectiveness, both the material and the shape of the incontinence insert are extremely important.

Since the range of currently available incontinence inserts for men is not yet satisfactory, many men rely on incontinence inserts intended primarily for women.—The incontinence insert for women has a shape to suit the anatomical conditions of the female genital region and thus is largely like the sanitary pads that are used for menstruation.—The shape of the pad of the incontinence insert is elongated; when made to adhere to the underwear, it protrudes prominently from the underwear and is poorly suited to use for male incontinence. Especially when a man moves, the penis, with its urethral orifice in the region of its tip, does not stay put on the female pad protruding prominently from the underwear; instead it tends to lie beside the pad, and thus does not remain in the center position as intended. In designing a incontinence insert for a man who is not completely incontinent, a man's habits in urination must also be taken into account.—For spontaneous urination, the woman pulls her underpants, with the pad sticking to them, all the way down and then urinates in a seated position. —Conversely, a man who is not completely incontinent tries to urinate while standing, as he is used to doing. If he is using the commercially available pads of the kind women use that are stuck to his underpants, he has to pull the top edge of his underpants down far enough that his penis is out in the open above the edge of the underpants so he can urinate while standing; naturally this often leads to soiling of his underwear and is not ideal.

The invention is intended primarily for the large number of men who do not have complete urinary incontinence. In this men, the bladder fills up to a certain extent without any release of urine through the urethra. As the bladder fills further, or if there is an uncontrollable urge to urinate, a release of urine occurs that the man can no longer suppress. For these men, it is important on the one hand that they be able to urinate quickly when the urge arises, or in other words, that the man can quickly get his penis free of his underwear and the pad, so he can urinate “normally”. On the other hand, the pad should absorb the urine that does escape despite all the man's intentional efforts and thus keep both his underwear and his body reliably dry.

The pads used by and recommended for men so far are largely equivalent in shape and material to the pads women use, merely being larger in size.—Incontinence inserts developed especially for men are usually based on a kind of bag whose underside is made to adhere to the underpants, and into which the penis is inserted from the top. The penis thus comes to rest with its underside on the upper edge of the bag. Disadvantage: Upon physical confirmation, the penis easily slips out of the relatively narrow bag, and is also hard to insert back into the bag again after urination. Moreover, the upper edge of the bag presses against the urethra, so that pressure damage with consequent urethra fistulas cannot be reliably precluded.

In the present invention, it has been assumed that to attain the object in question, that is, on the one hand reliable absorption of urine that has escaped unintentionally and on the other getting the penis quickly free for the sake of spontaneous urination, the point of departure must be a functional unit comprising an incontinence insert and underwear. As already noted, if there is a strong urge to urinate, the man can often hold back his urine for only a few seconds by using his voluntary bladder-closing muscle.—If he cannot get his penis free within this time, the unintentional escape of urine can no longer be prevented, and outside the incontinence insert, his underwear will become soiled.

According to claim 1, the secure position of the penis on the resorbent pad of the incontinence insert is assured by providing that the resorbent pad is equipped with peripheral beads, parallel to the longitudinal axis of the body. These peripheral beads are pressed against the abdominal wall when the pad is worn and together with the pad of the incontinence insert form a kind of channel in which the penis comes to rest, and from which it cannot come free even if the man makes extreme movements of his body.—When he has an urge to urinate, the man can easily free his penis from the channel by pulling down the upper edge of the underpants to which the pad is secured and bring his penis out into the open and begin spontaneous urination, and afterward he can easily place his penis back in the channel.—For special cases, the channel can also be closed at the top or bottom, or at both the top and bottom, in accordance with claims 2 and 3, with additional peripheral beads.—According to claim 4, part of the surface of the bead is resorbent.—Most of the incontinence inserts on the market are glued to the inside of the underpants, and some of the fabric between the upper end of the incontinence insert and the top edge of the underpants remains uncovered. This part of the underpants that remains uncovered can easily become soiled with urine, if the urge to urinate becomes uncontrollable while the penis is still being pulled out.—According to claims 5 and 6, the resorbent pad therefore has an upward-pointing extension, which is folded over the top edge of the underpants and secured to the front of the underpants. This extension preferably has a resorbent surface.—According to claim 7, for better sealing off of the channel, there is a downward-pointing extension of the pad, which is preferably trapezoidal, to suit anatomical conditions.—Another solution to the problem of quickly getting the penis free if the urge to urinate comes on suddenly is, according to claim 8, to provide a slit, located in the middle region of the pad, through which the penis can be pulled out.—Since the incontinence insert and the underpants are meant to form a functional unit, according to claims 9 and 10 these two elements are joined to one another by special devices in the region of the slit. For that purpose, the portion 1b in the region of the slit has extensions, which are joined to the slit region of the underpants.—The incontinence insert on the market comprises one resorbent component and one component that is impermeable to liquid, and these components are joined solidly and nondetachably to one another. According to claim 11, conversely, these components are two initially completely separate components, which are joined together only just before being put in place. This arrangement makes for a very economical variant. The impermeable portion can be used more than once, and the portion that is resorbent on all sides is very inexpensive to produce. The impermeable portion, in its shape, is in accordance with claims 1 and 2, but comprises a more-solid material than the more sheetlike one in the incontinence insert on the market, and is more like a relatively soft, elastic shell with peripheral beads, into the cavity of which the edges of the resorbent pad are secured through a gap, in accordance with claim 12.

Further advantages and characteristics of the invention can be learned not only from the further dependent claims, whose content is hereby expressly referred to, but also the ensuing description and the associated drawings of possible embodiments according to the invention. Shown in the essentially schematic drawings are:

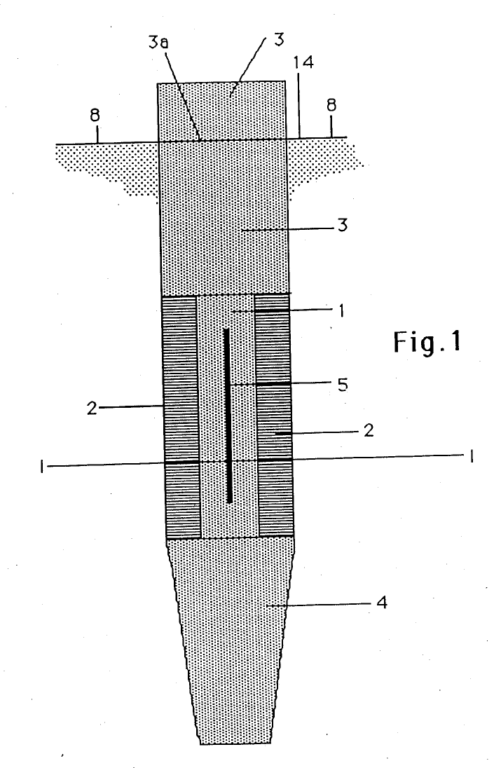

FIG. 1: a plan view on the incontinence insert, looking outward from the body;

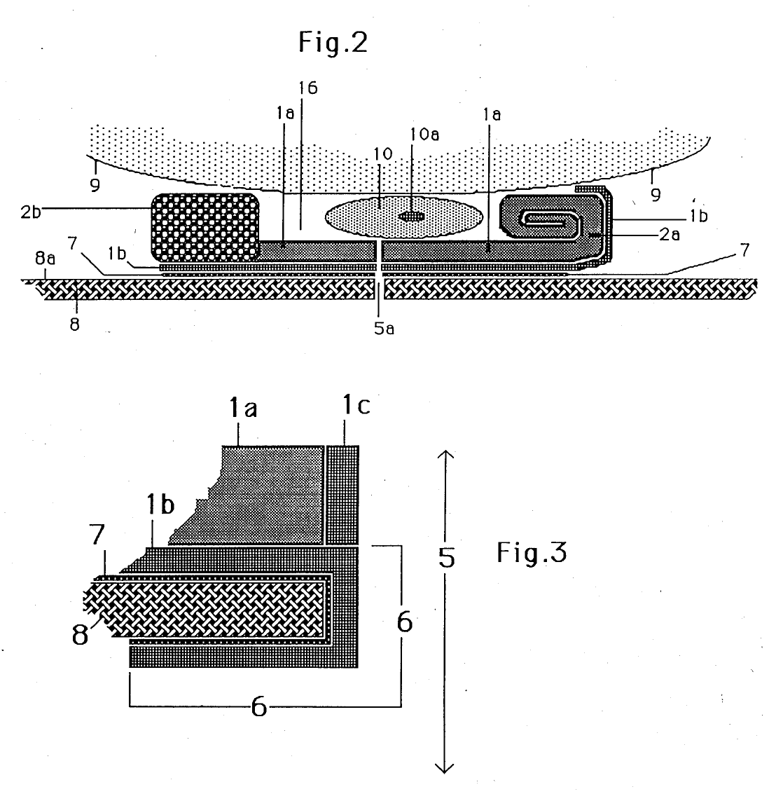

FIG. 2: a cross section through the incontinence insert taken along the line marked |- - - | in FIG. 1;

FIG. 3: an enlarged cross section of the region of the slit in FIG. 2;

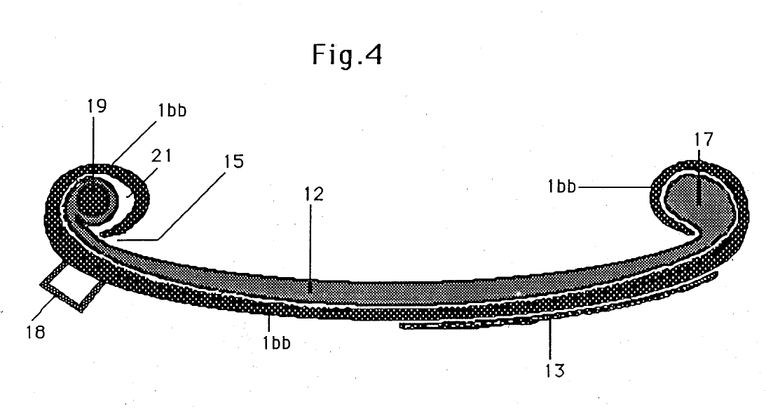

FIG. 4: a cross section through an incontinence insert of claims 11 and 12, with a pad in place that is resorbent on all sides.

In FIG. 1, one is looking outward from the body at the resorbent face of the pad 1, which when the pad is worn comes to rest on the surface of the abdominal wall and the penis between them. The urine-resorbent material takes up the urine escaping arbitrarily from the urethral orifice and keeps the surface of the abdominal wall and the underpants dry. This pad 1 is provided on both sides, parallel to the longitudinal axis of the body, with the peripheral beads 2, which assure that even if the man makes extreme movements, the penis will not move away. On the upper end is the extension 3 of the pad 1. This extension is folded forward over the top edge 14 of the underpants 8 in the region of the folding line 3a and fastened to the front of the underpants 3. The surface of the extension 3 is preferably resorbent.—The downward-pointing extension 4 of the pad 1 is shown here in trapezoidal form and likewise has a surface that is largely resorbent on the inside.—In the middle region of the pad, the longitudinal slit 5 is provided, through which the penis can be brought to the outside.

FIG. 2 shows a cross section of FIG. 1 along the line |- - - | and shows the relationships between the surface 9 of the abdominal wall, the pad 1 having the components 1a and 1b, the penis 10, the peripheral beads 2a and 2b, and the underpants 8. The two peripheral beads 2a and 2b rest on the abdominal wall surface 9, and together with the resorbent portion 1a of the pad 1 they form a channel 16, whose cavity disappears again when the pad is put on. In this channel the penis 10 rests immovably along with its urethral orifice 10a.—In this exemplary embodiment, the resorbent portion 1a of the pad 1 is solidly joined from the very outset to the impermeable portion 1b, and the portion 1b is detachably joined by the adhesive layer 7 to the inside surface 8a of the underpants. The impermeable portion 1b assures that no urine can escape from the channel 16 to reach the underpants 8.—The slit 5 or 5a of the underpants 8, through which the penis can be pulled out without having the pull down the upper edge of the underpants, is located in the middle region of the pad portions 1a and 1b. 2a is a bead, which was created by rolling up one edge of 1a and 1b and gluing it, while 2b is a roll of foam that is fastened to 1a.

FIG. 3 shows the enlarged edge of the right-hand region of the slit in FIG. 2. In this exemplary embodiment, the underpants 8 and pad portions 1a and 1b are functionally coupled together. 1b has the extension 6, which is folded over toward the outside around the slit edge of the underpants and fastened detachably by the adhesive layer 7. The impermeable portion 1c keeps the resorbent portion 1a from letting urine escape toward the slit 5.

FIG. 4 shows a cross section through an exemplary embodiment in which the two portions 1a and 1b of the pad 1, now called elements 12 and 1bb, are initially completely separate from one another. The two portions are not united until when they are to be worn. The element 12 has surfaces that are resorbent on all sides, and before being worn, it is introduced into the shell-like embodiment of 1bb. 1bb is embodied as a relatively soft, elastic shell with the raised features in accordance with claims 1 and 2. An edge, embodied in the manner of a bead, of 12 can be introduced through the gap 15 in the shell 1bb and clamped. by A connection can also be made between 12 and 1bb means of a cord or a rod as shown at 19. By means of the adhesive layer 13 or a hook-and-loop closure, the shell 1bb can be detachably fastened to the inside of the underpants. Reference numeral 18 indicates a retaining eyelet on the shell 1bb. A plurality of such eyelets serve to fasten the shell to the man's body, without requiring other fastenings to the underwear.

1. A device, comprising a pad 1, which on the side toward the body has a urine-absorbing portion 1a and on the side away from the body has a portion 1b that is impermeable to liquid, which portions are firmly joined together, and the pad can be attached detachably to both the body and the underwear,
characterized in that
the shape of the device is designed such that when the device is applied to the male genital region, the orifice 10a of the urethra always comes to rest in the region of the pad 1.

2. The device of claim 1, characterized in that

3. The device of claim 1, characterized in that the channel formed by the longitudinal beads 2 are closed by further beads at the top or bottom, or at the top and bottom.

4. The device of claim 1, characterized in that
inward, toward the pad, the peripheral beads have a liquid-resorbent surface.

5. The device of claim 1, characterized in that
the pad 1 has a resorbent surface pointing upward, which is dimensioned such that this extension can be folded over the top edge 14 of the underpants 8 in the region 3a and detachably fastened to the front side of the underpants.

6. The device of claim 5, characterized in that
the extension 3 of claim 5, in the region where it is folded over, is interrupted in impermeable fashion in its resorbent face, and the front side of the extension 3 is impermeable.

7. The device of claim 1, characterized in that
the pad 1 has a resorbent extension 4 extending downward, which is preferably embodied as trapezoidal or tapering in oval form.

8. The device of claim 1, characterized in that
in the middle region of the pad 1, there is a slit 5, though which the penis can be brought to the outside.

9. The device of claim 8, characterized in that
the impermeable portion 1b of the pad 1, in the region of the slit 5, has an extension 6 of impermeable material, which when inserted through the slit 8a in the underpants 8 keeps the edge of the slit in the underpants dry, and the extension 6 is detachably joined with the adhesive face 7 to the front side of the underpants.

10. The device of claim 8, characterized in that
in the region of the slit, the resorbent face 1a of the pad 1 is closed off from the slit 5 by the impermeable wall 1c.

11. A device, comprising a pad 1, which on the side toward the body has a urine-absorbing portion 1a and on the side away from the body has a portion 1b that is impermeable to liquid,
characterized in that
the two portions are put together to make a functional unit only immediately before the device is to be worn, and the portion 1b is embodied here in the form of the portion 1bb, comprising a softer, elastic plastic material corresponding to the shape of 1b of claims 1 and 1, that is, has longitudinal beads on both sides, and the pad 12 that is resorbent on all sides is inserted into this portion.

12. The device of claim 11, characterized in that
the fixation of the pad 12 that is resorbent on all sides with the peripheral reinforcement is effected by being clamped under the elastic edges of the peripheral beads with the gap 15 of 1bb.

13. The device of claim 11, characterized in that
the longitudinal edges of the pad 12 that is resorbent on all sides are reinforced with a rod or a cord 19, such that they can be inserted longitudinally into the cavity 21 in the longitudinal beads of 1bb and thus fixed.

14. The device of claim 1, characterized in that
the shapes need not always be rectangular, but can instead have rounder or oval forms.
